(19) United States
(12) Patent Application Publication
(10) Pub. No.: US 2004/0176465 A1
(43) Pub. Date: Sep. 9, 2004

(54) Solid compositions containing fluoxetine and a coating

(76) Inventor: Pawan Seth, Irvine, CA (US)

Correspondence Address: HARNESS, DICKEY & PIERCE, P.L.C., P.O. BOX 828, BLOOMFIELD HILLS, MI, 48303, US

(21) Appl. No.: 10/384,185

(22) Filed: Mar. 7, 2003

(51) Int. Cl. A61K 31/137; A61K 9/24

(52) U.S. Cl. 514/649; 424/471

(57) ABSTRACT

The invention provides a solid composition comprising fluoxetine, exhibiting a dissolution profile, as determined in a basket at 100 rpm and a buffer pH 7.5, such that after 15 minutes, from 30 to 70% of the fluoxetine is released, after 30 minutes, from 70 to 98% of the fluoxetine is released, after 45 minutes, more than 90% of the fluoxetine is released.

### BACKGROUND OF THE INVENTION

The present invention relates to novel solid compositions, notably pharmaceutical compositions, containing fluoxetine and a coating, and to methods for their preparation.

Fluoxetine, esp. its HCl salt, is a known antidepressant, marketed under the name Prozac®. There is a need now to obtain specific release profiles, with manufacturing processes which are easy to carry out.

### SUMMARY OF THE INVENTION

The present invention provides a solid fluoxetine composition comprising, by weight based on the total weight of the composition:

(a) a tablet core comprising, by weight based on the core weight, from 30 to 80% of fluoxetine; and

(b) a coating comprising, based on the weight of the coating, from 50 to 90% of a gastroresistant polymer;

exhibiting a dissolution profile, as determined in a basket at 100 rpm and a buffer pH 7.5, such that after 15 minutes, from 30 to 70% of the fluoxetine is released, after 30 minutes, from 70 to 98% of the fluoxetine is released, after 45 minutes, more than 90% of the fluoxetine is released.

### DETAILED DESCRIPTION

The expression “solid composition” means that the composition is in a tablet or mini-tablet form, these in their turn being able to be encapsulated using for example the conventional hard gelatin. In one embodiment, the composition is comprised of tablets in capsule, e.g. a hard gelatin capsule size 1. Usually, the tablet core comprises, by weight based on the core weight, from 30 to 80% of fluoxetine.

According to one embodiment, in the core of the composition according to the invention, the core comprises conventional additives such as microcrystalline cellulose, lactose, ascorbic acid, pigments, plastifying agents, lubricants, fillers, disintegrating agents such as cross-linked PVP, water-soluble polymers such as PVP, and so on. Obviously, other conventional additives known to those skilled in the art can be employed.

According to one embodiment of the invention, the core comprises magnesium stearate and/or glycerol behenate and/or sodium stearyl fumarate, which is employed as a lubricant ensuring better compression of the core when provided in tablet form, for example. The composition of the invention is provided with a coating. This coating comprises, based on the weight of the coating, from 50 to 90% of a gastroresistant polymer. The gastroresistant polymer withstands the acidic medium of the stomach and the duodenum, but will dissolve in the intestines, as soon as the pH reaches a predetermined level (e.g. above 5.5 or above 7). This gastroresistant polymer can be selected from the group consisting in (uncured) poly(meth)acrylic acid, cellulose and alkylcellulose-phtalates. Molecular weight can vary within broad limits as will recognize the skilled man. The term “uncured” is used to differentiate over U.S. Pat. No. 5,580,578.

Preferably, it is of the type of Eudragit® L30D55. One preferred polymer is an anionic copolymer on the basis of methacrylic acid and acrylic acid ethyl ester. The formula is as follows:

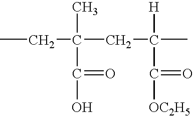

The ratio free carboxyl group to ester group is preferably about 1:1. The mean molecular weight is e.g. about 250,000. Such a copolymer will easily dissolve at pH values above 5.5 with the forming of salts.

The coating may further comprise polyethyleneglycol, present in an amount from 3 to 20% by weight, based on the total weight of the functional coating. Stearic acid, dibutyl sebacate, propylene glycol and/or triethyl citrate can used in lieu of or in addition to polyethyleneglycol as plasticizers. The coating may also comprise other excipients, such as talc. The coating usually represents from 5 to 20% by weight of the core weight, esp. about 10%.

The present composition can be obtained by the following process, comprising the steps of:

(i) granulating fluoxetine and excipients into granules;

(ii) compressing the granules obtained at step (i) into tablet cores;

(ii) coating said tablet cores obtained at step (ii) with a gastroresistant polymer.

The granulating step may be comprised of the following sub-steps:

(a) mixing in the dry state and for a sufficient time, fluoxetine and one or several additives;

(b) adding a solution of a water-soluble polymer, followed by mixing for a sufficient period of time;

(c) drying the wet granules thus formed for a sufficient period of time; and

(c) optionally sieving the thus-obtained granules.

The solvent employed, when use is made of a solvent, is preferably water. The choice of mixing times, apparatus used, sieve mesh, and other operating conditions are within the province of the normal knowledge of those skilled in the art.

The composition may be further additionally coated. Surface coating is employed for the purposes of improving appearance making the drug more readily acceptable to the patient, or for dimensionally stabilizing the compressed tablet. The coating can be a conventional coating suitable for enteral use. It is obtained using any conventional technique employing conventional ingredients. A surface coating can for example be obtained using a quick-dissolving film.

The examples below are provided as examples illustrating the invention and should not be considered as limiting its scope.

### EXAMPLE

The following composition was prepared:

| Ingredient | mg |
|---|---|
| Core | 100.60 |
| Fluoxetine HCl | 50.00 |
| Avicel PH 101 | 10.00 |
| Plasdone K25 | 1.00 |
| Magnesium stearate | 13.00 |
| Water | 50.00 |
| Coating | |
| Eudragit L30D55 | 10.80 |
| Carbowax 1450 granular | 1.08 |
| Talc (Lo micron) | 4.32 |
| Water | 105.00 |

The core manufacturing process is as follows. The fluoxetine and microcrystalline cellulose are introduced into the granulator bowl and dry mixed for 2.00 minutes (High shear granulator KG5, key international). The PVP solution is then added and the mass kneaded for 3.50 minutes at 450 rpm. The mass is then dried in a fluid bed dryer (Glat CPGC1) at 55° C. for 20 minutes. The dried granules are screened through a 0.045″ screen. The polyplasdone and Mg stearate are then added to the granules and blend for 10 minutes in PVK Blender. The final granulation mixture is compressed into tablet using 5.46 mm diameter round punches. The tablet properties are: thickness 4.0 mm, 5.5 KP.

The coating manufacturing process is as follows. PEG is dissolved into 30% of the water. Eudragit is added and stirred gently using a paddle stirrer. Talc is suspended in the remaining water. The suspension is added to the Eudragit solution. The resulting composition is filtrated and coating is performed in a perforated pan. After completion of the coating the tablets are dried 30 minutes. The apparatus used is a Vector LCDS with the following spraying parameters.

| | |
|---|---|
| Nozzle: | 1.2 mm |
| Atomizing air | 20 psi |
| Air volume | 70 cfm |
| Inlet Temperature | 40° C. |
| Spraying Rate | 7.5 g/min |

The dissolution profile has been determined (basket 100 rpm, USP buffer pH 7.5). The results are the following.

| | Time (min) | | | | | | | | |
|---|---|---|---|---|---|---|---|---|---|
| | 0 | 15 | 30 | 45 | 60 | 90 | 120 | 150 | 180 |
| % | 0.00 | 45.07 | 90.88 | 95.93 | 97.80 | 99.05 | 99.40 | 99.60 | 99.67 |
| dis- | | | | | | | | | |
| solved | | | | | | | | | |

While various embodiments of the pharmaceutical composition and method have been disclosed, it should be appreciated that variations may be made to the present invention. It is intended by the following claims to cover these and any other departures from the disclosed embodiments which fall within the true spirit of this invention.

The invention claimed is:

1. A solid fluoxetine composition comprising, by weight based on the total weight of the composition:
(a) a tablet core comprising, by weight based on the core weight, from 30 to 80% of fluoxetine; and
(b) a coating comprising, based on the weight of the coating, from 50 to 90% of a gastroresistant polymer;
exhibiting a dissolution profile, as determined in a basket at 100 rpm and a buffer pH 7.5, such that after 15 minutes, from 30 to 70% of the fluoxetine is released, after 30 minutes, from 70 to 98% of the fluoxetine is released, after 45 minutes, more than 90% of the fluoxetine is released.

2. The composition according to claim 1, in which, in the coating, the gastroresistant polymer is comprised of an anionic copolymer on the basis of methacrylic acid and acrylic acid ethyl ester.

3. The composition according to claim 1, in which the coating further comprises polyethyleneglycol, present in an amount from 3 to 20% by weight, based on the total weight of the functional coating.

4. The composition according to claim 1, in which the core further comprises microcrystalline cellulose, PVP, cross-linked PVP and magnesium stearate.

5. The composition according to claim 1, in which the core is comprised of granules compressed together.

6. A solid fluoxetine composition comprising, by weight based on the total weight of the composition:
(a) a tablet core comprising, by weight based on the core weight, from 30 to 80% of fluoxetine, where the core is comprised of granules compressed together; and
(b) a coating comprising, based on the weight of the coating, from 50 to 90% of a gastroresistant polymer which is comprised of an anionic copolymer on the basis of methacrylic acid and acrylic acid ethyl ester and from 3 to 20% by weight of polyethyleneglycol;
exhibiting a dissolution profile, as determined in a basket at 100 rpm and a buffer pH 7.5, such that after 15 minutes, from 30 to 70% of the fluoxetine is released, after 30 minutes, from 70 to 98% of the fluoxetine is released, after 45 minutes, more than 90% of the fluoxetine is released.

7. The composition according to claim 6, in which the core further comprises microcrystalline cellulose, PVP, cross-linked PVP and magnesium stearate.

8. The composition according to claim 1, in a capsule.

9. The composition according to claim 6, in a capsule.

10. A process for the manufacture of a fluoxetine solid composition, comprising the steps of:
(i) granulating fluoxetine and excipients into granules;
(ii) compressing the granules obtained at step (i) into tablet cores, where the tablet core comprises, by weight based on the core weight, from 30 to 80% of fluoxetine; and
(ii) coating said tablet cores obtained at step (ii) with a gastroresistant polymer, where the coating comprises, based on the weight of the coating, from 50 to 90% of a gastroresistant polymer;
wherein the thus-obtained fluoxetine solid composition exhibits a dissolution profile, as determined in a basket at 100 rpm and a buffer pH 7.5, such that after 15 minutes, from 30 to 70% of the fluoxetine is released, after 30 minutes, from 70 to 98% of the fluoxetine is released, after 45 minutes, more than 90% of the fluoxetine is released.

11. The process of claim 10, in which the granulating step (i) comprises the following sub-steps:
(a) mixing in the dry state and for a sufficient time, fluoxetine and one or several additives;
(b) adding a solution of a water-soluble polymer, followed by mixing for a sufficient period of time;
(c) drying the wet granules thus formed for a sufficient period of time; and
(c) optionally sieving the thus-obtained granules.
